(19) United States
(12) Patent Application Publication
(10) Pub. No.: US 2022/0023110 A1
(43) Pub. Date: Jan. 27, 2022

(54) ABSORBENT ARTICLES HAVING ABSORBENT LEG CUFFS

(71) Applicant: ATTENDS HEALTHCARE PRODUCTS, INC., Raleigh, NC (US)

(72) Inventors: Charles F. SCHROER, Jr., Raleigh, NC (US); Harry CHMIELEWSKI, Raleigh, NC (US)

(73) Assignee: ATTENDS HEALTHCARE PRODUCTS, INC., Raleigh, NC (US)

(21) Appl. No.: 17/311,055

(22) PCT Filed: Dec. 3, 2019
(86) PCT No.: PCT/US2019/064136

§ 371 Date: Jun. 4, 2021

Related U.S. Application Data
(60) Provisional application No. 62/775,434, filed on Dec. 5, 2018.

(51) Int. Cl. A61F 13/494; A61F 13/49

(52) U.S. Cl. CPC A61F 13/49426; A61F 13/49017; A61F 2013/49092; A61F 13/49473; A61F 13/4942

(57) ABSTRACT

This disclosure includes absorbent articles comprising first and second absorbent cuffs to prevent leaks. The present articles can comprise a chassis having opposing front and rear portions and a crotch portion extending longitudinally between the front and rear portions. An absorbent core can extend longitudinally along the crotch portion, and the first and second absorbent cuffs can extend longitudinally along the crotch portion as well. The absorbent cuffs can be positioned such that at least a portion of the absorbent core is positioned between the absorbent cuffs, and each can comprise a liftable portion and one or more elastics configured to urge the liftable portion away from the chassis and toward a wearer when the absorbent article is worn. Absorbent or hydrophobic end caps can extend across the ends of the absorbent cuffs.

### CROSS-REFERENCE TO RELATED APPLICATIONS

This application claims priority to U.S. Provisional Application No. 62/775,434, filed Dec. 5, 2018, the contents of which is incorporated into the present application in its entirety.

### FIELD OF INVENTION

The present invention relates generally to absorbent articles and, more particularly, to absorbent articles having absorbent leg cuffs for leak prevention.

### BACKGROUND

Absorbent products can include, for example, baby diapers, training pants, and adult incontinence briefs and underwear, bladder control pads, feminine hygiene pads, pet diapers and pads, etc., all of which may be made in disposable forms. “Disposable” refers to articles that are designed to be discarded after a limited use rather than being laundered or otherwise restored for reuse. Disposable absorbent products have met with widespread acceptance in the marketplace for a variety of applications, including infant and adult incontinence care, in view of the manner in which such products can provide effective and convenient liquid absorption and retention while maintaining the comfort of the wearer. Such disposable absorbent articles often include a topsheet that is configured to be closest to the wearer during use, a liquid-impermeable backsheet or outer cover, and an absorbent core between the topsheet and the backsheet. In some instances, such disposable absorbent articles also include an acquisition-distribution layer (ADL) disposed between the topsheet and the absorbent core. Hydrophobic, elasticated standing leg cuffs and leg gathers are also often used in such articles to provide improved fit and reduced leakage around a wearer's legs, relative to articles without such cuffs or gathers.

U.S. Pat. No. 4,670,011 discloses certain prior art examples of diapers, and U.S. Pat. Nos. 6,976,978 and 4,940,464 disclose certain prior art examples of disposable incontinence garments or training pants.

One example of such a disposable absorbent article is shown in FIGS. 1A-1B, which depict a lower plan view and a perspective view, respectively, of training pant 10. Training pant 10 includes a chassis 14 having a front waist portion 18, an opposing rear waist portion 22, and a crotch portion 26 extending longitudinally between front and rear waist portions 18, 22. Chassis 14 further includes a backsheet 30 defining an outer surface and configured to face away from a wearer during use of the training pant, and topsheet 34 defining an opposing body facing surface and configured to face a wearer during use of the training pant.

As shown in FIGS. 1A-1B, training pant 10 further includes a pair of front elastic side panels 38 and a pair of rear elastic side panels 42 configured to couple rear waist portion 22 to front waist portion 18 in a well-known configuration in which a left side 46 of the chassis defines a first leg opening 50 for a wearer's left leg, and in which a right side 54 of the chassis defines a second leg opening 58 for the wearer's right leg. In the depicted configuration, each of side panels 38, 42 includes a connection portion 62 configured to be coupled to a connection portion 62 of another of side panels 38, 42. Specifically, connection portion 62 of the left one of front side panels 38 is configure to be coupled to connection portion 62 of the left one of rear side panels 42, and connection portion 62 of the right one of front side panels 38 is configure to be coupled to connection portion 62 of the right one of rear side panels 42, such that the waist portions 18, 22 and side panels, 38, 42 cooperate to define a waist opening 66 as shown in FIG. 1B. Connection portions 62 of the respective side panels can be permanently coupled together to define a tear-able side seam 70, such as, for example, via adhesive, ultrasonic, or thermal bonds. Such tear-able side seams generally cannot be refastened, and thereby render an article unusable once opened. Alternatively, connection portions 62 of the respective side panels can be removably coupled to define a refastenable or adjustable side seam, such as, for example, via hook-and-loop fasteners. Hook and loop fasteners are mechanical fasteners that include hooks, such as in a hook fastener portion, that are configured to engage loops in a loop fastener portion or in fibers of a sheet of fabric; for example, a nonwoven or woven fabric with fibers that define open or loop-like regions into which the hooks can extend and engage. Examples of such hook and loop fasteners may be referred to as VELCRO®.

As is known in the art, training pant 10 can include one or more elastic elements coupled to the chassis such that the one or more elastic elements resist expansion of a circumference of the first leg opening and resist expansion of a circumference of the second leg opening. For example, as shown in FIG. 1A, the depicted embodiment of chassis 14 includes a first elastic region 74 along right side 46, and a second elastic region 78 along left side 54. In some configurations, elastic regions 74, 78 can each be defined by one or more elastic strands, which may be referred to in the art as “leg elastics,” coupled to the chassis, for example laminated between the topsheet or an additional leg cuff layer and the backsheet. In other configurations, elastic regions 74, 78 can each be defined by an elastic film coupled to the chassis, for example laminated between the topsheet and the backsheet. In configurations in which elastic regions 74, 78 are defined by elastic film, the regions can be defined by separate pieces of elastic film or by separate regions of a single piece of elastic film. As shown in FIG. 1A, elastic regions 74, 78 may be parallel to and/or extend along a majority of a length of each of sides 46 and 54, provided that the elastic regions are configured to provide a biasing force that resists expansion of the leg openings when the chassis is in its closed configuration and tends to contract the leg opening around a wearer's leg, as shown in FIG. 1B. Contraction of the leg opening to conform to the wearer's leg is desired for containment of urine and feces in an absorbent product.

Another example of such a disposable absorbent article is shown in FIGS. 2A and 2B, which depict lower plan views of a baby diaper 100. Diaper 100 includes a chassis 104 having a front waist portion 108, an opposing rear waist portion 112, and a crotch portion 116 extending longitudinally between front and rear waist portions 108, 112. Chassis 104 further includes an outer surface 128 configured to face away from a wearer during use of the diaper, and an opposing body facing surface 132 configured to face a wearer during use of the diaper. In the view of FIG. 2A, a dashed leader extends from the body facing surface to reference numeral 132 because body facing surface 132 is opposite outer surface 128 and therefore not visible in the view of FIG. 2A.

As shown in FIG. 2A, diaper 100 further includes a pair of closure members 136 configured to couple rear waist portion 112 to front waist portion 108 in a well-known configuration in which a left side 140 of the chassis defines a first leg opening for a wearer's left leg, and in which a right side 144 of the chassis defines a second leg opening for the wearer's right leg, similar in some respects to what is shown in FIG. 1B for training pant 10. In the depicted configuration, the closure members include a pair of back ears or back ear panels 148 each having a first end 152 bonded to rear waist portion 112 of chassis 104, and a second end 156 shown extending away from rear waist portion 112. “Bonded” refers to the joining, adhering, connecting, attaching, or the like, of two elements via adhesive(s), ultrasonic bond(s), and/or thermal bond(s). Two elements will be considered to be bonded together when they are bonded directly to one another or indirectly to one another, such as when each is directly bonded to intermediate elements.

Each closure member 136 further includes a fastener tab 160 with a first end 164 bonded to back ear 148, a second end 168 shown extending laterally outward from back ear 148, and a fastener portion 172 coupled to the fastener tab. Back ears 148 are each formed of a stretchable elastic material, such as a nonwoven laminate, that permits adjustments in the width and tension of back ears 148 to vary the form and fit of diaper 100 when worn by a user.

Fastener tabs 160 are formed of an inelastic nonwoven material and carry fastener portions 172. Fastener portions 172 include strips of hook material configured to interact with a corresponding loop material in the well-known hook-and-loop fastener arrangement. Connection of closure members 136 to front waist portion 108 is facilitated by a landing zone 176 configured to be engaged by fastener portions 172. In this embodiment, landing zone 176 is defined by an anchoring member that includes a strip of loop material bonded to front waist portion 108 of chassis 104, for example, to the backsheet, and configured to be engaged by the hook material of fastener portions 172.

As shown in FIG. 2A, diaper 100 also includes a pair of front ears 180 extending from opposite sides 140, 144 of chassis 104 with each of front ears 180 each having a first end 184 bonded to front waist portion 108 of chassis 104, and a second end 188 shown extending away from a respective side of front waist portion 108. Front ears 180 are each formed of a relatively soft nonwoven material and are each configured to be overlapped by the corresponding fastener tab 160 and/or back ear 148 to prevent the edges of fastener tab 160 from pinching, rubbing, or otherwise irritating a user's skin in use when fastening portions 172 are engaged with landing zone 176 to couple rear waist portion 112 to front waist portion 108.

Outer surface 128 is defined by a liquid-impermeable backsheet or cover 192 that defines outer surface 128, and a liquid-permeable topsheet 196 that defines body facing surface 132 and is configured to be closest to the wearer during use. “Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. “Lamination” is the technique of manufacturing a material in multiple layers, so that the composite material has benefits of all the combined layers, such as, for example, improved mechanical strength or durability, improved stability, lower permeability to water, and/or other properties. A laminate includes two or more layers of material(s) that are permanently assembled by heat, pressure, ultrasonic welding, or adhesives.

As shown in FIG. 2B, the depicted embodiment include an absorbent core 200 disposed between topsheet 196 and backsheet 192. An “absorbent core” is a structure typically disposed between a topsheet and backsheet of an absorbent article and containing materials like SAP and/or cellulosic fibers that are configured to absorb liquid in the absorbent article.

As shown in FIG. 2B, diaper 100 also includes an acquisition-distribution layer (ADL) 204 disposed between the topsheet and the absorbent core. “Layer” when used in the singular can be a single element or a plurality of elements. For example, a plurality of sheets may together define a single layer, such as, for example, a layer with a particular function to which the sheets of the layer contribute.

As is known in the art, diaper 100 can include one or more elastic elements coupled to the chassis such that the one or more elastic elements resist expansion of a circumference of the first leg opening, a circumference of the second leg opening, and/or a circumference of the waist opening. For example, as shown in FIG. 2B, the depicted configuration of chassis 104 includes a first elastic region 208 along first side 140, a second elastic region 208 along second side 140, a third elastic region 208 along front waist portion 108, and a fourth elastic region 208 along rear waist portion 112. In some configurations, elastic regions 208 can each be defined by one or more elastic strands, which may be referred to in the art as “leg elastics” or “leg gathers” along first and second sides 140 and 144 and “waist elastics” or a “waistband” along front and rear waist portions 108 and 112, coupled to the chassis, for example laminated between the topsheet (or an additional leg cuff layer) and the backsheet. In other configurations, elastic regions 208 can each be defined by an elastic film coupled to the chassis, for example laminated between the topsheet (or an additional leg cuff layer) and the backsheet. In configurations in which elastic regions 208 are defined by elastic film, the regions can be defined by separate pieces of elastic film or by separate regions of a single piece of elastic film, such as in a waistband. As shown in FIG. 2B, elastic regions 208 may be parallel to and/or extend along a majority of a length of each of sides 140 and 144 and front and rear waist portions 108 and 112, provided that the elastic regions are configured to provide a biasing force that resists expansion of the leg openings and waist opening when the chassis is in its closed configuration.

As shown in FIG. 2A, chassis 104 has an overall relaxed length 212.

The standing leg cuffs of conventional absorbent articles (e.g., training pant 10 and diaper 100) are typically constructed of a hydrophobic nonwoven material to repel and thereby contain liquid insults. Hydrophobic leg cuffs generally must form a seal with a wearer's skin to contain liquid insults; liquid and/or feces may escape if the seal is broken, resulting in undesirable leakage. Conventional leg cuffs often cannot maintain an adequate seal due to, for example, movement of the wearer and/or the accumulation of liquids and/or feces in the absorbent article. Conventional absorbent articles thus may not provide adequate leak prevention for the wearer. There is a need in the art for absorbent articles that can better contain liquids and/or feces.

### SUMMARY

The present absorbent articles address the need for improved liquid and/or feces containment with absorbent leg cuffs configured to define a barrier that absorbs and contains liquids and semi-solids. The leg cuffs can absorb liquid insults to reduce accumulation thereof and thereby maintain a seal or liquid barrier with the wearer's skin. In some embodiments, the leg cuffs can absorb any liquid that breaks and escapes past the seal to further reduce leakage out of the article. And, in some embodiments, the leg cuffs can have a folded configuration that can provide an improved seal or liquid barrier with the wearer's skin.

Some of the present absorbent articles comprise a chassis having opposing front and rear portions and a crotch portion extending longitudinally between the front and rear portions. Some articles comprise an absorbent core that extends longitudinally along the crotch portion.

Some articles comprise first and second absorbent cuffs. In some articles, the cuffs extend longitudinally along the crotch portion and, optionally, are positioned such that at least a portion of the absorbent core is positioned between the absorbent cuffs. In some articles, each of the absorbent cuffs comprises a liftable portion and, optionally, one or more elastics configured to urge the liftable portion away from the chassis and toward a wearer when the absorbent article is worn. In some articles, the one or more elastics comprise a plurality of elastic strands extending longitudinally along the absorbent cuff. The elastic strands, in some articles, include two or more outer elastic strands and an inner elastic strand disposed closer to a longitudinal center axis of the absorbent article than are the outer elastic strands. In some articles, the outer elastic strands are laterally spaced apart from each other by a first separation distance and the inner elastic strand is laterally spaced apart from an innermost one of the outer elastic strands by a second separation distance. In some articles, the second separation distance is at least 50% larger than the first separation distance. The innermost outer elastic strand, in some articles, is disposed closer to the longitudinal center axis than are the other outer elastic strand(s). The spacing of the inner and outer elastic strands can at least in part determine how each of the cuff(s) lifts away from the chassis, including whether the cuff stands up in a manner similar to traditional cuffs (e.g., a “stand-up cuff” configuration) or in a manner in which at least a portion of the cuff is comparatively flatter relative to a wearer's skin to improve the seal therebetween (e.g., a “gasketing cuff” configuration). In some articles, each of the absorbent cuffs has first and second bonded ends, optionally a bonded base, and a free portion extending longitudinally between the bonded ends, wherein the bonded ends are attached to the chassis and the free portion is unattached to the chassis. The free portion, in some articles, is configured to lift away from the chassis, e.g., to define a stand-up cuff or a gasketing cuff.

In some articles, each of the cuffs comprises a laminate having two or more substrate laminae and one or more absorbent laminae. In some of such articles, a first one of the absorbent lamina(e) is disposed between first and second ones of the substrate laminae. In some articles, the first absorbent lamina comprises superabsorbent polymer (SAP), at least one of the first and second substrate laminae comprises a nonwoven, and/or at least one of the first and second substrate laminae comprises tissue. Each of the absorbent cuffs, in some articles, is longitudinally folded such that the absorbent cuff includes a lower layer and one or more folded layers disposed above the lower layer.

In some articles, the first and second absorbent cuffs are separate, e.g., each of the absorbent cuffs is separate from the other of the absorbent cuffs. Other articles comprise a cuff assembly or insert that has an absorbent body that defines the first and second absorbent cuffs. The absorbent body, in some articles, extends longitudinally along the crotch portion and comprises first and second body surfaces. In some articles, the absorbent body is longitudinally folded such that the cuff assembly includes a base that spans a lateral width of the cuff assembly. The base, in some articles, has a lower surface defined by a portion of the first body surface and an upper surface defined by a portion of the second body surface. The absorbent body, in some articles, is longitudinally folded such that the cuff assembly defines a longitudinally-extending center region disposed between first and second longitudinally-extending edge regions, wherein within each of the edge regions, the body defines the lower layer and the folded layer(s) of one of the absorbent cuffs. In some articles, the elastic(s) are coupled to the second body surface.

Some articles comprise a nonwoven underlayer coupled to the lower surface of the base. In some articles, for each of the absorbent cuffs, the underlayer extends along the first body surface and onto the second body surface such that the elastic(s) are disposed between the second body surface and the underlayer. Other articles, for each of the absorbent cuffs, comprise a nonwoven cover layer having a first edge coupled to the first body surface in the edge region and a second edge coupled to the second body surface such that the cover layer extends along the first body surface and onto the second body surface and the elastic(s) are disposed between the second body surface and the cover layer. In some articles, the underlayer has a first basis weight and the cover layer has a second basis weight that is heavier than the first basis weight.

Some articles comprise first and second nonwoven over-layers, each extending longitudinally along the crotch portion and partially disposed on the absorbent core such that an uncovered portion of the absorbent core is defined between the over-layers. In some of such articles, the center region of the cuff assembly is attached to the uncovered portion. In some articles, the over-layers cover at least a portion of the absorbent core.

In some articles, the absorbent core comprises one or more core laminates, each comprising two or more substrate laminae and, optionally, one or more absorbent laminae. In some of such articles, a first one of the absorbent lamina(e) comprises SAP and, optionally, is disposed between first and second ones of the substrate lamina(e). In some articles, the one or more core laminates comprise two or more core laminates. A first one of the core laminates, in some articles, has a first length and a first width. In some articles, a second one of the core laminates has a second length shorter than the first length and/or a second width larger than the first width. In some of such articles, each of the lengths is measured longitudinally and each of the widths is measured laterally. In some articles, the second core laminate at least partially overlies or underlies the first core laminate and, optionally, is positioned closer to the front portion than is the first core laminate.

In some articles, the first core laminate is longitudinally folded such that the absorbent core includes a lower layer of the first core laminate that spans a lateral width of the absorbent core and one or more folded layers of the first core laminate disposed above the lower layer. The folded layers, in some articles, are disposed within each of first and second longitudinally-extending core edge regions, each of the core edge regions spanning less than 50% of the width. In some of such articles, the second core laminate is disposed on the lower layer of the first core laminate.

In some articles each of the absorbent cuffs extends longitudinally between opposing first and second ends and the absorbent article comprises first and second end caps. The first end cap, in some articles, is attached to the front portion of the chassis and extends laterally across the first ends of the absorbent cuffs. The second end cap, in some articles, is attached to the rear portion of the chassis and extends laterally across the second ends of the absorbent cuffs. At least one of the first and second end caps, in some articles, comprises a free portion that at least partially overlies and is unattached to the absorbent cuffs. In some articles, at least one of the first and second end caps comprises one or more elastic strips, each configured to urge the free portion of the end cap away from the chassis and toward a wearer when the absorbent article is worn. In some articles, each of the first and second end caps comprises the laminate of the absorbent cuffs. Each of the first and second end caps, in some articles, comprises an absorbent material.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the term “substantially” may be substituted with “within [a percentage] of” what is specified, where the percentage includes 0.1, 1, 5, and 10 percent.

The terms “comprise” and any form thereof such as “comprises” and “comprising,” “have” and any form thereof such as “has” and “having,” and “include” and any form thereof such as “includes” and “including” are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

The term “nonwoven,” as used herein, and per an INDA definition, refers to sheet or web structures bonded together by entangling fiber or filaments, and/or by perforating films, mechanically, thermally, or chemically. They are flat, porous sheets that are made directly from separate fibers or from molten plastic or plastic film. They are not made by weaving or knitting and do not require converting the fibers to yarn. The term “film” refers to a membrane-like layer of material formed of one or more polymers, which does not have a form consisting predominately of a web-like structure of fibers and/or other fibers.

The term “liquid impermeable,” when used in describing a material, means that a liquid, such as urine, will not pass through the material, under ordinary use conditions, in a direction generally perpendicular to the plane of the material at the point of liquid contact. The term “breathable,” when used in describing a material, means that the material has a water vapor transmission rate (“WVTR”) of at least about 300 grams/m²/24 hours. “Breathable” materials can be substantially liquid impermeable.

The term “superabsorbent,” “superabsorbent material,” “superabsorbent polymer,” or “SAP” refers to a water-swellable, water-insoluble organic or inorganic material capable, under the most favorable conditions, of absorbing at least about 15 times its weight in an aqueous solution containing 0.9 weight percent sodium chloride and, more desirably, at least about 30 times its weight in an aqueous solution containing 0.9 weight percent sodium chloride and, even more desirably, at least about 50 times its weight in an aqueous solution containing 0.9 weight percent sodium chloride. The SAP materials used in the present methods and articles can be natural, synthetic and modified natural polymers and materials. In addition, the SAP materials can be or include organic compounds such as cross linked polymers. “Cross-linked” is a commonly understood term and refers to any approach for effectively rendering normally water-soluble materials substantially water insoluble, but swellable. Such polymers can include, for example, carboxymethylcellulose, alkali metal salts of polyacrylic acids, polyacrylamides, polyvinyl ethers, hydroxypropyl cellulose, polyvinyl morpholinone, polymers and copolymers of vinyl sulfonic acid, polyacrylates, polyacrylamides, polyvinyl pyridine and the like. Other suitable polymers can include hydrolyzed acrylonitrile grafted starch, acrylic acid grafted starch, and isobutylene maleic anhydride copolymers, and mixtures thereof. Organic high-absorbency materials can include natural materials, such as agar, pectin, guar gum and peat moss. In addition to organic materials, the SAP materials may also include inorganic materials, such as absorbent clays and silica gels. The SAP material can comprise fibers and/or particles that may be spherical, spherical-like or irregularly shaped particles, such as sausage shaped particles, or ellipsoid shaped particles of the kind typically obtained from inverse phase suspension polymerizations. The SAP particles can also be optionally agglomerated at least to some extent to form larger irregular particles. In some embodiments, the SAP particles can also have a surface modification, such as a partial or full surface coating, for example to increase the hydrophilicity of the SAP particles.

Exemplary superabsorbent polymer material suitable for use in the present methods and articles can comprise any superabsorbent polymer particles known from superabsorbent literature, for example such as described in Modern Superabsorbent Polymer Technology, F. L. Buchholz, A. T. Graham, Wiley 1998. Suitable examples of SAP include T9030, T9600, T9900, and Saviva polymers from BASF Corporation in Charlotte, N.C.; and W211, W112A, W125, S125D, QX-W1482, QX-W1486, QX-W1504, and QX-W1505 from Nippon Shokubai Co. Ltd, N.A.I.I. in Houston, Tex.; and AQUA KEEP SA50 II, SA55SX II, SA60N II, SA65s, HP500, HP500E, HP600, and HP 700E from Sumitomo Seika Chemicals Co., Ltd. in Osaka, Japan.

As used herein, the terms absorbent core, core, and containment core can be used interchangeably.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Further, a device or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Each of the figures is drawn to scale, unless otherwise noted, meaning the sizes of the depicted elements are accurate relative to each other for at least the depicted embodiment.

FIG. 1A is a bottom plan view of a prior art disposable absorbent article, specifically a training pant, in an open configuration.

FIG. 1B is a perspective view of the training pant of FIG. 1A in a closed configuration.

FIG. 2A is a bottom plan view of a prior art disposable absorbent article, specifically a baby diaper, in an open configuration.

FIG. 2B is a bottom plan view of the baby diaper of FIG. 2A, in an open configuration, showing certain internal components of the diaper.

FIG. 3A is a top view of a first embodiment of the present articles that comprises first and second absorbent leg cuffs. FIG. 3A represents the portions of the cuffs that are disposed under the end caps with dashed lines. A partial outline of the absorbent core disposed under the topsheet is also represented with dashed lines.

FIG. 3B is a sectional view of the article of FIG. 3A taken along line 3B-3B and illustrates the folded construction of the absorbent leg cuffs.

FIG. 3C is an enlarged, partial sectional view of the article of FIG. 3A that illustrates the folded construction of the leg cuffs, the configuration of the laminate that forms the leg cuffs, and the spacing of the elastic strands that urge the leg cuffs toward a wearer when the article is worn.

FIG. 3D is a sectional view of the article of FIG. 3A when the article is in a wearable configuration in which the leg cuffs lift away from the chassis.

FIG. 3E is a sectional view of the article of FIG. 3A taken along line 3E-3E when the article is in a wearable configuration. FIG. 3E depicts one of the end caps of the article when the end cap is lifted away from the chassis to define a dam configured to receive liquids and/or feces.

FIGS. 4A and 4B are sectional views of a second embodiment of the present absorbent articles in which the leg cuffs are defined by a cuff assembly.

FIG. 5 is a sectional view of a third embodiment of the present absorbent articles that, for each of the leg cuffs, has a cover layer that is folded over a free edge of the cuff and secures the elastic strands.

FIG. 6A is a top view of a fourth embodiment of the present absorbent articles having an absorbent core that comprises first and second core laminates, wherein the second core laminate is wider and shorter than the first core laminate.

FIG. 6B is a sectional view of the article of FIG. 6A taken along line 6B-6B. FIG. 6B illustrates the relative widths of the first and second core laminates.

FIG. 7 is a sectional view of a fifth embodiment of the present absorbent articles having an absorbent core that comprises a first core laminate that is folded and a second core laminate layered on the first core laminate.

FIG. 8 is a sectional view of a sixth embodiment of the present absorbent articles in which the absorbent cuffs are defined by and are integral with a core laminate of the absorbent core.

FIGS. 9A and 9B are top and side views, respectively, of a mannequin used to perform an absorbency-before-leakage test to compare the leakage performance of some of the present absorbent articles to that of a standard hydrophobic cuff diaper.

FIGS. 9C-9E illustrate a diaper applied to the mannequin of FIGS. 9A and 9B during the absorbency-before-leakage test. FIG. 9C illustrates the pouch formed by the diaper between the legs of the mannequin and proper positioning of the leg elastics, FIG. 9D illustrates proper positioning of the diaper's cuffs relative to the mannequin's male adapter, and FIG. 9E illustrates the diaper's tapes secured to the landing zone thereof.

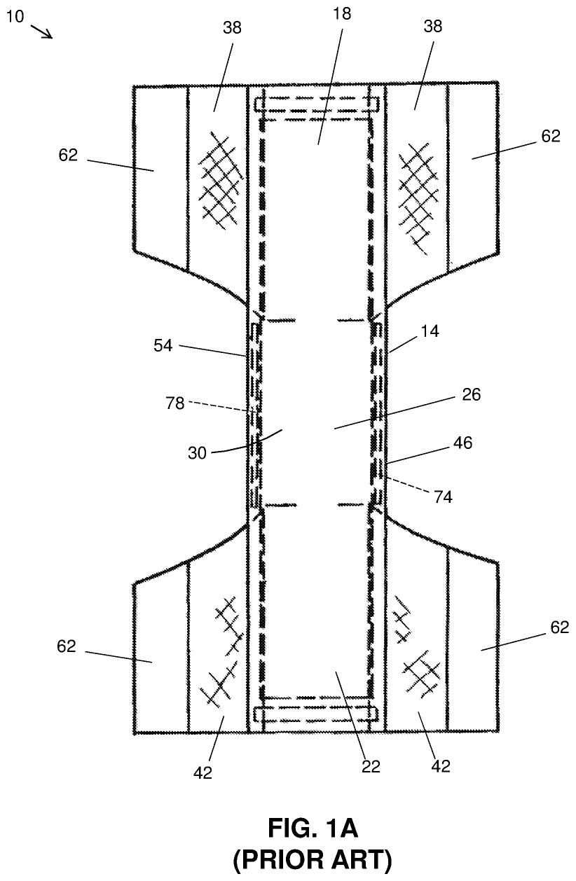

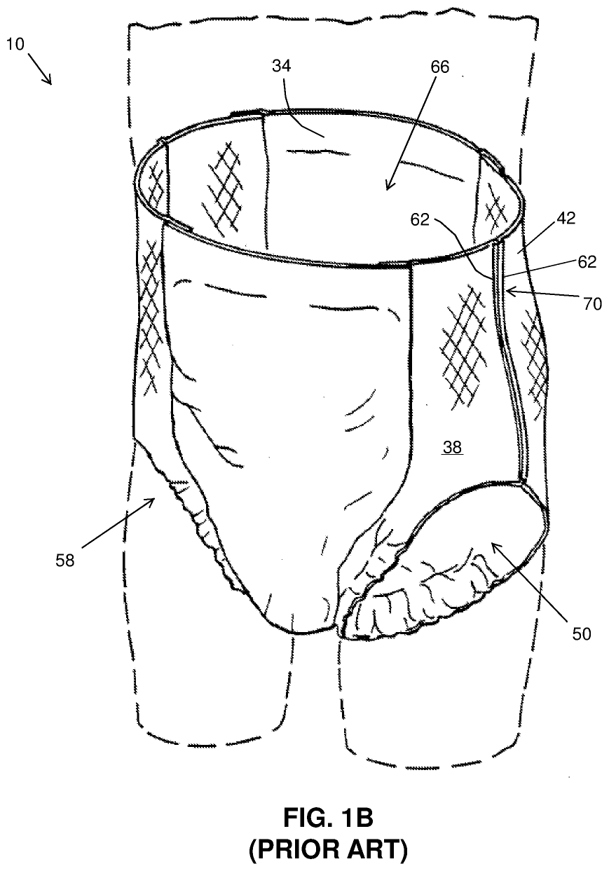

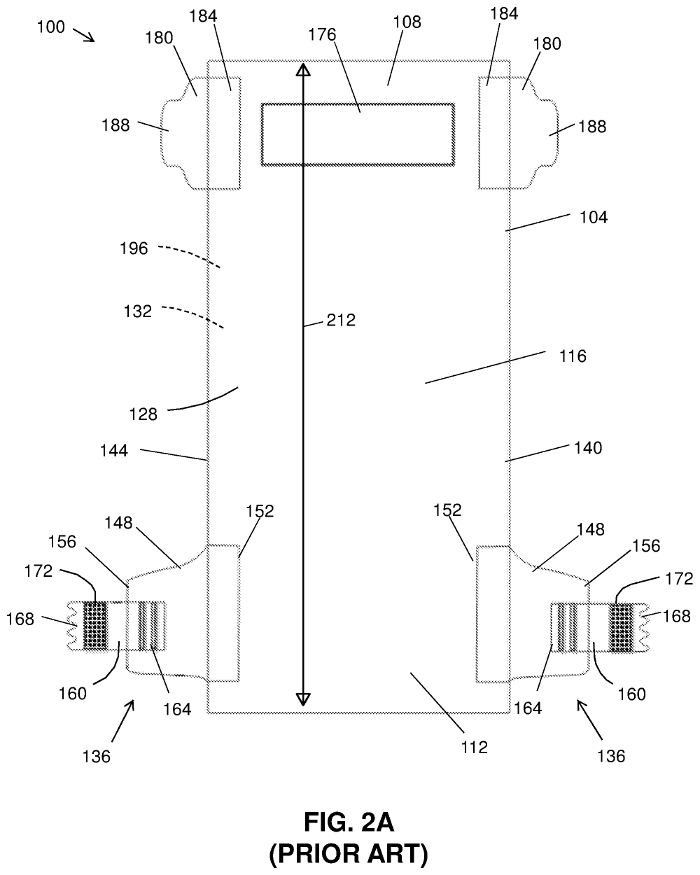

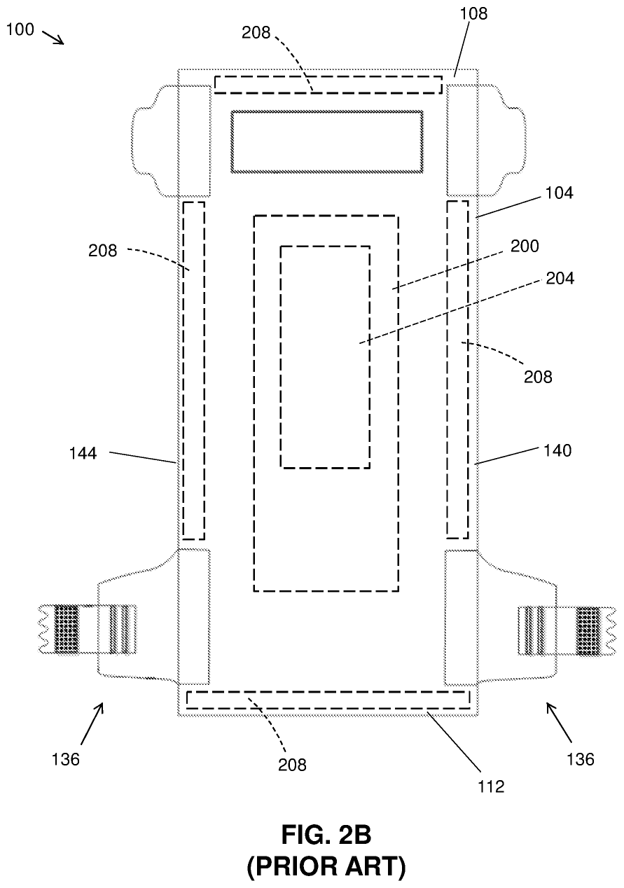

### DETAILED DESCRIPTION OF ILLUSTRATIVE EMBODIMENTS

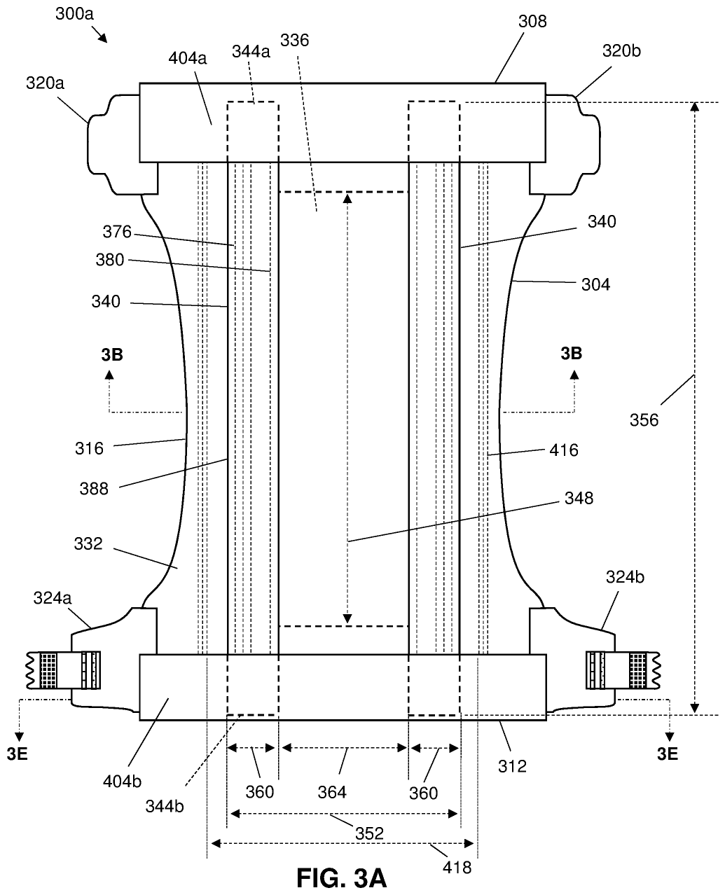

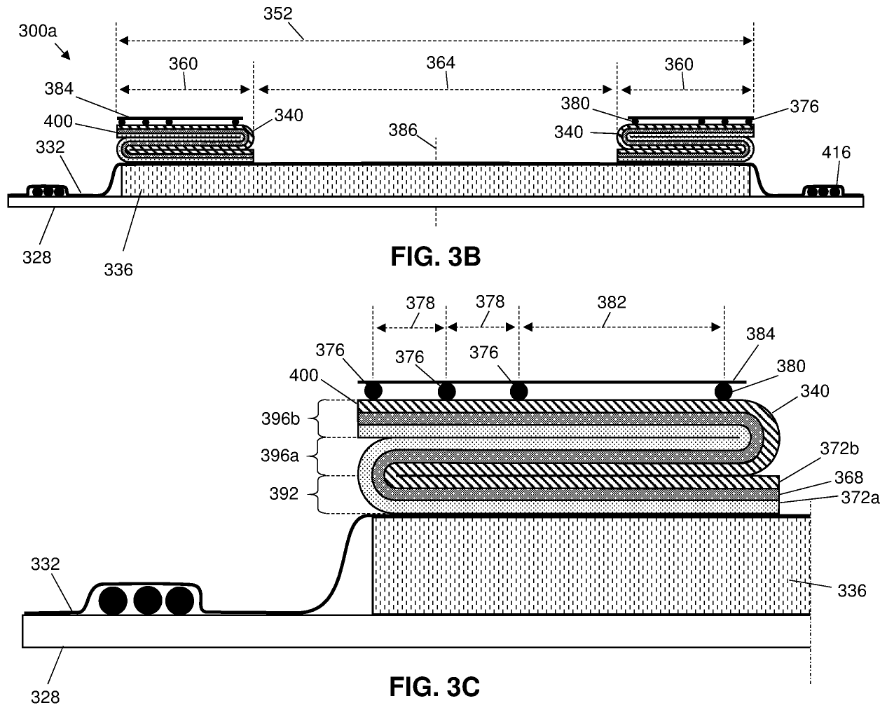

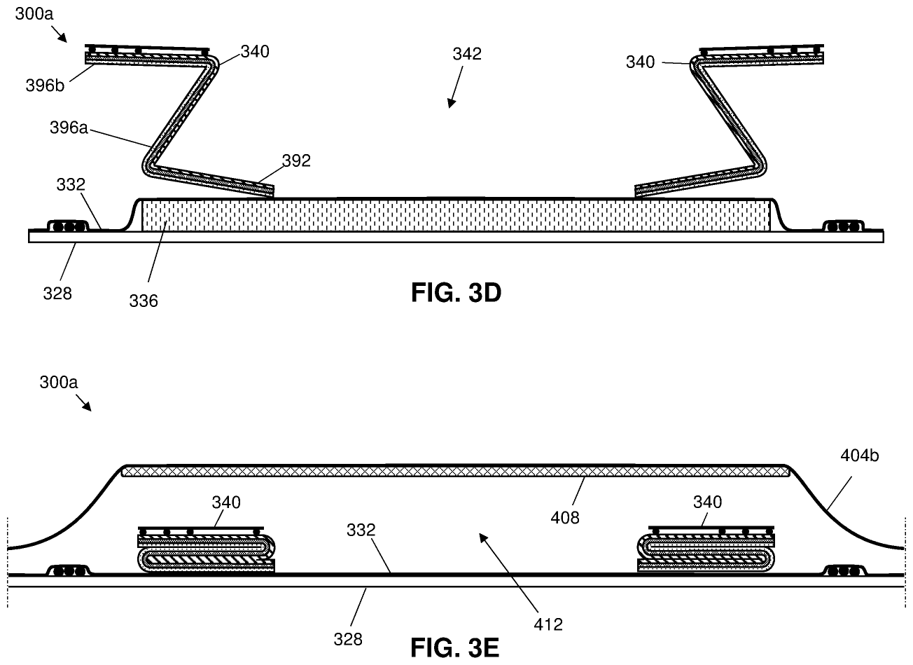

Referring to FIGS. 3A-3D, shown is an embodiment 300a of the present absorbent articles. Article 300a can be a baby diaper, training pant, adult incontinence brief or underwear, bladder control pad, feminine hygiene pad, or the like, and comprises a chassis 304. Chassis 304 can have a crotch portion 316 that extends longitudinally between a front portion 308 and a rear portion 312. Chassis 304 can define an extended configuration (FIG. 3A) (e.g., an open configuration if the article is a diaper, training pant, incontinence brief, or the like) and a wearable configuration (e.g., a closed configuration if the article is a diaper, training pant, incontinence brief, and the like) in which crotch portion 316 is configured to conform about the groin area, perineum, and/or buttocks of a wearer. For example, hook portions of back ears 324a and 324b can encircle a wearer's waist and engage a loop material of a landing zone 320a and 320b on front portion 308 or nonwoven fibers of backsheet 328.

Chassis 304 can have a backsheet 328 configured to face away from a wearer and, optionally, a liquid-permeable topsheet 332 configured to face the wearer during use of article 300a. Backsheet 328 can be liquid-impermeable and can include, for example, an inner liquid-impermeable film and an outer nonwoven backsheet that can be a nonwoven fabric. In some articles, backsheet 328 can be breathable, for example, an inner liquid-impermeable film of the backsheet can comprise a breathable film. Article 300a can include an absorbent core 336 that extends longitudinally along crotch portion 316. Core 336 can, but need not, extend longitudinally along the entire length of crotch portion 316. For example, core 336 can have a longitudinal length 348 that is less than the entire chassis length but is greater than or equal to, or between any two of, 25%, 35%, 45%, 55%, 65%, or more (e.g., greater than or equal to 25%) of the length of the entire chassis. Article 300a can further comprise, along each of the longitudinal edges of chassis 304 in crotch portion 316, a set of one or more leg gather elastics 416 disposed between backsheet 328 and topsheet 332. Gather elastics 416 can provide an improved fit. Core 336 can be positioned laterally inboard of each of the sets of gather elastic(s) 416, e.g., a lateral width 352 of the core can be smaller than (e.g., less than or equal to 95% of) a minimum separation distance 418 between each of the sets of gather elastic(s).

Core 336 can comprise any material or combination of materials suitable for absorbing liquids, such as, for example, fluff, SAP, and/or one or more laminates, described in further detail below. As shown, absorbent core 336 is disposed between backsheet 328 and topsheet 332 and comprises a mixture of fluff and SAP.

Article 300a can comprise first and second absorbent cuffs 340 that extend longitudinally along crotch portion 316 and are configured and positioned to promote containment of liquid and/or fecal insults. At least a portion of each of absorbent cuffs 340 can be configured to lift away from chassis 304 toward a wearer (FIG. 3D). For example, each of absorbent cuffs 340 can comprise one or more elastics (e.g., 376 and 380) that urge the cuff into the lifted configuration when article 300a is worn. Absorbent cuffs 340 can each have a lateral width 360 that is smaller than a lateral width 352 of absorbent core 336 and can be laterally spaced apart by a cuff separation distance 364 such that at least a portion, optionally all, of the absorbent core is positioned between the absorbent cuffs. As used herein, at least a portion of the absorbent core can be “positioned between” the absorbent cuffs whether or not that portion (or the entire absorbent core) is coplanar with the absorbent cuffs. As a result, absorbent cuffs 340, when lifted, can define a containment area 342 therebetween that promotes liquid and/or fecal containment for absorption by core 336.

To illustrate, each of absorbent cuffs 340 can be disposed on absorbent core 336 along a respective one of the longitudinal edges thereof such that width 352 of the absorbent core is within 10% of the combined width of cuff widths 360 and separation distance 364. In other embodiments, each of absorbent cuffs 340 can be disposed laterally outboard of a respective one of the longitudinal edges of absorbent core 336 and can, but need not, have a portion that overlies the absorbent core (e.g., separation distance 364 can be smaller than or equal to width 352 of the absorbent core); in other embodiments, all of the core can be disposed between the cuffs (e.g., separation distance 364 can be larger than width 352). Core 336 can extend laterally to each of the sets of gather elastic(s) 416, e.g., width 352 can be within 10% of separation distance 418 between the sets of gather elastic(s), and/or can extend laterally outboard of cuffs 340 such that width 352 is larger than the combined width of cuff widths 360 and separation distance 364. Each of cuffs 340 can be sized appropriately relative to core 336 to promote adequate lifting and seal formation with a wearer's skin. For example, each of cuff widths 360 can be less than or equal to, or between any two of, 80%, 70%, 60%, 50%, 40%, 30%, 20%, 10%, or less of core width 352, such as, for example, less than or equal to or between any two of 60 mm, 50 mm, 40 mm, 30 mm, 20 mm, 10 mm, or less (e.g., less than or equal to 50 mm).

Each of absorbent cuffs 340 can extend into front portion 308 (optionally to the front end of the chassis) and/or rear portion 312 (optionally to the rear end of the chassis) (e.g., first end 344a of the cuff can be disposed within the front portion, optionally at the front chassis end, and second end 344b of the cuff can be disposed within the rear portion, optionally at the rear chassis end). Absorbent cuffs 340 can thereby facilitate liquid and/or feces containment along substantially all of the length of article 300a. Core 336 can, but need not, have a longitudinal length 348 that is shorter than length 356 of each of absorbent cuffs 340 (e.g., such that the absorbent core does not extend to front and rear portions 308 and 312). A comparatively shorter absorbent core can reduce the bulk of article 300a. Absorbent cuffs 340, by defining a containment area 342 that extends longitudinally beyond absorbent core 336, can facilitate containment and delivery of liquids to the absorbent core even if those liquids do not initially contact the core.

Unlike conventional cuffs—which function by temporarily containing, but not absorbing, liquids—absorbent cuffs 340 can absorb and contain liquids and semi-solids that remain outside the absorbent core and contact the cuff. Optionally, each of absorbent cuffs 340 can be configured to absorb liquids that escape containment area 342, in addition to liquids disposed therein. Absorbent cuffs 340 can thereby mitigate leakage even if liquids break the seal between the cuffs and a wearer's skin due to each of the cuffs being absorbent on both the inside and outside portions of the cuff. In other embodiments, however, each of absorbent cuffs 340 can comprise a liquid-impermeable material, e.g., a liquid-impermeable film, on a surface of the cuff disposed outside of containment area 342 (e.g., to facilitate containment when the cuff has reached its absorption capacity and cannot absorb more liquid) or on a surface of the cuff disposed within the containment area (e.g., such that the cuff absorbs liquid that escapes past the liquid-impermeable film to reduce leakage).

Absorbent cuffs 340 can comprise any suitable absorbent material that is configurable to lift away from chassis 304 and define containment area 342, such as, for example, an absorbent laminate and/or a nonwoven containing a superabsorbent material (e.g., a nonwoven that is impregnated by SAP particles and/or comprises superabsorbent fibers). As shown, each of absorbent cuffs 340 comprises a laminate having one or more substrate laminae (e.g., 372a and 372b) and one or more absorbent laminae (e.g., 368). Each of the absorbent lamina(e) can comprise SAP particles and, optionally, the SAP particles can be disposed within a matrix of adhesive material. Suitable adhesive material can include, for example, a thermoplastic hot-melt adhesive composition or a pressure-sensitive thermoplastic adhesive composition. For example, absorbent lamina 368 can comprise at least 90% (e.g., greater than 93% or 94%), by weight, SAP and less than or equal to 10% (e.g., less than 6% or 7%), by weight, adhesive. To illustrate, absorbent lamina 368, including the SAP, can have a basis weight of at least 10 grams per square meter (gsm), such as, for example, greater than or equal to or between any two of 10, 20, 30, 40, 50, 60, or more gsm and, optionally, less than or equal to 50 gsm (e.g., between 10 and 40 gsm).

Each of the substrate lamina(e) can be constructed from nonwoven material and/or tissue. Suitable nonwoven materials can include, for example, spunbond, spunlace, or carded webs of one or more polymers, including polypropylene, polyethylene, nylon, polyester, and blends of these materials. When constructed from a nonwoven, a substrate lamina can have a basis weight of at least 7 gsm, such as, for example, a basis weight greater than or equal to, or between any two of, 10, 20, 30, or more gsm and, optionally, less than or equal to 50 gsm (e.g., between 10 and 40 gsm). Suitable tissues can include, for example, porous tissues, creped tissues, and standard tissues. When constructed from tissue, a substrate lamina can have a basis weight of at least 10 gsm, such as, for example, a basis weight greater than or equal to, or between any two of, 10, 12, 14, 16, 18, 20, 22, 24, 26, 28, 30, or more gsm and, optionally, less than or equal to 50 gsm (e.g., between 10 and 30 gsm). The comparatively lighter basis weights of the absorbent lamina(e) and substrate lamina(e), as listed above, can permit absorbent cuffs 340 to lift away from chassis 304 and promote comfort for the user.

The substrate lamina(e) can facilitate liquid acquisition and distribution throughout cuffs 340 to reduce liquid accumulation in containment area 372 and thereby mitigate leakage. SAP in the absorbent lamina(e) swells when it absorbs liquid, which can impede liquid distribution throughout the laminate (referred to as “gel blocking”). Low-permeability SAP can minimize fluid movement into and through cuffs 340 to mitigate gel blocking, but high-permeability SAP may in some instances be preferred for quicker fluid transfer. Tissue and/or nonwoven substrate lamina(e) can mitigate gel blocking by promoting the acquisition and distribution of liquid throughout the absorbent lamina(e). To illustrate, the laminate of each of cuffs 340 can have a first substrate lamina 372a constructed from tissue, a second substrate lamina 372b constructed from a nonwoven, and an absorbent lamina 368 comprising at least 90% SAP by weight. Absorbent lamina 368 can be in contact with and disposed between first substrate lamina 372a and second substrate lamina 372b (FIG. 3C). As configured, when each of cuffs 340 is lifted, nonwoven second substrate lamina 372b can face containment area 342 to absorb and distribute rapid insults of liquid to absorbent lamina 368 and thereby reduce liquid accumulation in the containment area. First substrate lamina 372a, when constructed from tissue, can provide a capillary network through which liquid is spread to absorbent lamina 368 to mitigate gel blocking and thereby maintain an adequate acquisition rate to further reduce liquid accumulation.

In other embodiments, the laminate of each of absorbent cuffs 340 can have any suitable number of substrate and absorbent laminae arranged in any suitable order, such as, for example, greater than or equal to or between any two of 1, 2, 3, 4, 5, 6, 7, 8, or more substrate laminae and greater than or equal to or between any two of 1, 2, 3, 4, 5, 6, 7, 8, or more absorbent laminae. The number of substrate and absorbent laminae can be selected such that the overall basis weight of the laminate does not appreciably limit the liftability of cuffs 340. To illustrate, the total basis weight of the laminate can be less than or equal to or between any two of 200 gsm, 180 gsm, 160 gsm, 140 gsm, 120 gsm, 100 gsm, 80 gsm or less (e.g., less than or equal to 125 gsm or between 100 gsm and 150 gsm). Any two adjacent laminae in the laminate can be the same type of laminae (e.g., both can be substrate laminae or absorbent laminae) or laminae of different types (e.g., one can be one of the substrate lamina(e) and one can be one of the absorbent lamina(e)). By way of illustration, the laminate of each of absorbent cuffs 340 can comprise three substrate laminae and two absorbent laminae arranged such that each of the absorbent laminae is disposed between two of the substrate laminae. Providing additional absorbent laminae can increase the absorption capacity of cuffs 340. Providing additional substrate laminae can increase the strength of cuffs 340, the ability of cuffs to transfer and distribute fluid, among other properties.

Absorbent cuffs 340 can have any construction suitable for forming a seal or barrier with a wearer's skin. For example, each of absorbent cuffs 340 can be longitudinally folded one or more times such that the cuff includes a lower layer (e.g., 392) and one or more folded layers (e.g., 396a and 396b) disposed above the lower layer (e.g., a “gasketing cuff” configuration). As shown, each of absorbent cuffs 340 comprises a Z-fold, e.g., the cuff has two folded layers 396a and 396b disposed on lower layer 392, wherein, optionally, the uppermost one of the folded layers (e.g., 396b) comprises an outwardly-facing free edge 400 of the cuff (e.g., an edge facing away from longitudinal center axis 386 of article 300a). Each of cuffs 340 can partially unfold and expand upwards away from chassis 304 such that the cuff, to lift and form a seal with the skin of a wearer, need not tilt upward as much as cuffs having an unfolded construction. As a result, for each of cuffs 340, the angle between the cuff's seal-forming surface and a wearer's skin can be comparatively smaller such that a comparatively larger cuff surface area is in contact with the skin, thereby forming an improved seal. While cuffs 340, as shown, each comprise a Z-fold, in other embodiments the cuffs can be folded any suitable configuration to define any suitable number of folded layers, such as, for example, greater than or equal to or between any two of 1, 2, 3, 4, 5, 6, 7, 8, or more folded layers. In some embodiments, each of cuffs 340 can have an unfolded construction (e.g., can comprise a single layer) having any known “stand-up cuff” construction (e.g., a construction in which the cuff is configured to lift away from chassis 304 solely by pivoting) in which the cuff comprises any of the absorbent materials describe herein. Optionally, article 300a can comprise more than two absorbent cuffs that are substantially similar to cuffs 340, such as for example, greater than or equal to or between any two of 2, 4, 6, 8, 10, 12, or more cuffs.

Each of cuffs 340 can be attached to chassis 304 to maintain the cuff's folded configuration while permitting a portion of the cuff to lift away from the chassis. For example, each of first and second ends 344a and 344b can be attached to chassis 304 at front and rear portions 308 and 312, respectively. A free portion 388 disposed between first and second ends 344a and 344b can be unattached to chassis 304 and absorbent core 336 such that the free portion remains liftable. Optionally, the lower layer and folded layer(s) can be attached to one another at first and second ends 344a and 344b to maintain the folded configuration of the cuff. However, in other embodiments, at least a portion of each of cuffs 340 (e.g., lower layer 392) disposed between first and second ends 344a and 344b can be attached to chassis 304 and/or absorbent core 336. Attachments can be achieved via any suitable means, such as, for example, an adhesive or an ultrasonic bond.

Each of cuffs 340 can have one or more elastics (e.g., 376, 380) configured to urge the liftable portion of the cuff away from chassis 304 when article 300a is worn. As shown, the elastic(s) comprise a plurality of elastic strands extending longitudinally along cuff 340. The elastic strands can comprise, for example, two or more outer elastic strands 376 and an inner elastic strand 380 that is disposed closer to longitudinal center axis 386 of article 300a than are the outer elastic strands (e.g., adjacent an innermost one of the longitudinal folds). Outer elastic strands 376 can be laterally spaced apart from each other by a first separation distance 378 and inner elastic strand 380 can be laterally spaced apart from an innermost one of the outer elastic strands by a second separation distance 382. Second separation distance 382 can be larger than first separation distance 378, such as, for example at least or between any two of 50%, 60%, 70%, 80%, 90%, 100%, 110%, 120%, 130%, 140%, or more larger than the first separation distance. To illustrate, first separation distance 378 can be less than or equal to, or between any two of, 10 mm, 9 mm, 8 mm, 7 mm, 6 mm, 5 mm, 4 mm, 3 mm, 2 mm, or less (e.g., less than or equal to 6 mm) and second separation distance 382 can be greater than or equal to, or between any two of, 5 mm, 6 mm, 7 mm, 8 mm, 9 mm, 10 mm, 11 mm, 12 mm, 13 mm, 14 mm, 15 mm, or more (e.g., greater than or equal to 9 mm). Positioning elastic strands 376 and 380 in this manner can promote proper positioning of cuff 340 when the cuff is lifted such that the cuff forms an improved seal with a wearer's skin, as described above. Elastic strands 376 and 380 can be attached to an upper surface of cuff 340 (e.g., to an uppermost one of the folded layer(s)) and a cover layer 384 can be disposed over the elastic strands, e.g., to secure the elastic strands between the upper cuff surface and the cover layer. Cover layer 384 can comprise, for example, a nonwoven.

Referring additionally to FIG. 3E, article 300a can comprise first and/or second end caps 404a and 404b. First and second end caps 404a and 404b can mitigate leakage from front and rear portions 308 and 312. First end cap 404a can be attached to front portion 308 and can extend laterally across first ends 344a of cuffs 340, and second end cap 404b can be attached to rear portion 312 and can extend laterally across second ends 344b of the cuffs. Each of end caps 404a and 404b can have a free portion that at least partially overlies and is unattached to cuffs 340 and/or absorbent core 336. End caps 404a and 404b can thereby be configured to lift away from chassis 304 and toward a wearer when article 300a is worn. When lifted, each of end caps 404a and 404b can define a dam 412 configured to receive liquids and/or feces to mitigate leakage out of front and rear portions 308 and 312. Ends caps 404a and 404b can each have an elastic strip 408 (e.g., a foam elastic strip) configured to urge the end cap into the lifted configuration. In other embodiments, however, ends caps 404a and 404b can be secured to chassis 304 such that the end caps are not liftable. End caps 404a and 404b can comprise any suitable material, such as, for example, a nonwoven or an absorbent material (e.g., any of the above-described absorbent materials). When constructed from an absorbent material, end caps 404a and 404b can absorb and contain liquids and/or semi-solids that are not absorbed by core 336 to promote containment along front and rear portions 308 and 312. Additionally or alternatively, at least one of end caps 404a and 404b can comprise a hydrophobic material. In some embodiments, each of end caps 404a and 404b can have a construction substantially similar to that of cuffs 340 (e.g., a folded, “gasketing cuff” construction or a “stand-up cuff” construction). For example, each of end caps 404a and 404b can comprise one of the present laminates and can be laterally folded such that the end cap defines a lower layer of the laminate and one or more folded layers of the laminate disposed above the lower layer.

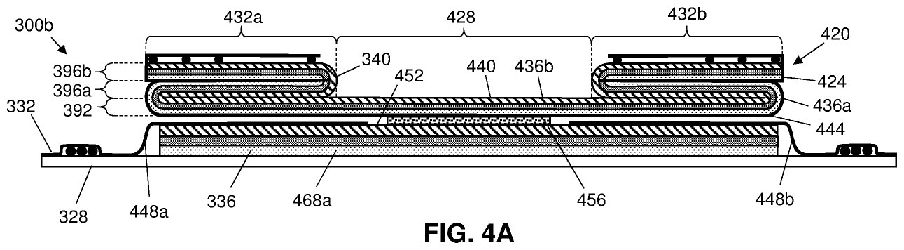

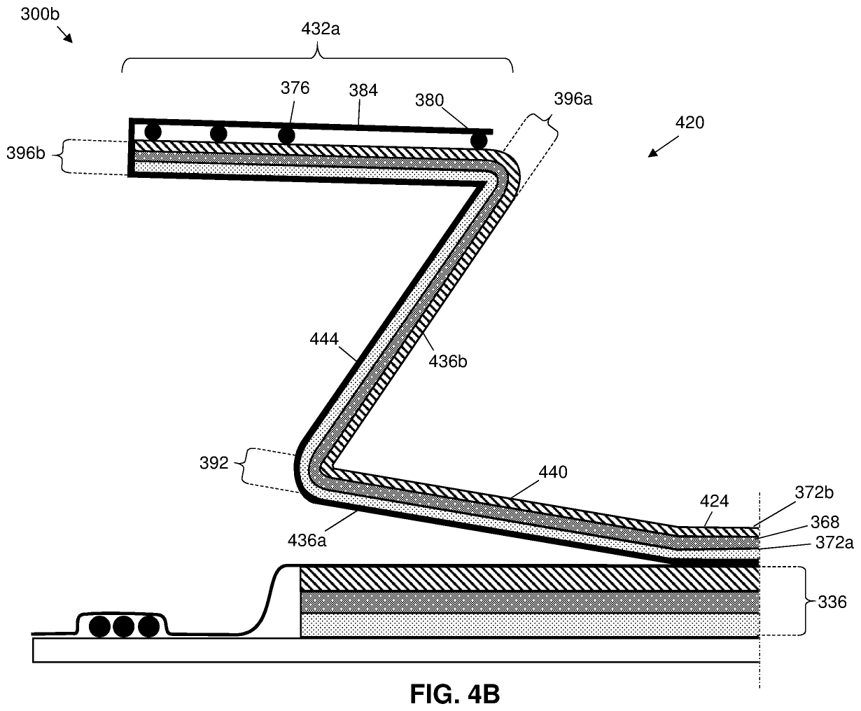

While article 300a, as shown, comprises two separate cuffs 340, in other embodiments the cuffs can be defined by a single assembly or “insert.” Referring to FIGS. 4A and 4B, shown is an absorbent article 300b that is substantially similar to article 300a; article 300b, however, can comprise a cuff assembly or “insert” 420 having an absorbent body 424 that defines both first and second cuffs 340. Body 424 can comprise any of the absorbent materials described above with respect to cuffs 340; as shown, for example, the body comprises the above-described absorbent laminate. Body 424 can have first and second body surfaces 436a and 436b (e.g., defined by first substrate lamina 372a and second substrate lamina 372b, respectively) and extend longitudinally along crotch portion 316. As shown, cuff assembly 420 can have a longitudinally-extending center region 428 disposed between first and second longitudinally-extending edge regions 432a and 432b. Body 424 can be longitudinally folded such that cuff assembly 420 includes a base 440 that spans a lateral width of the cuff assembly and, within each of edge regions 432a and 432b, the body defines the lower layer (e.g., 392) (e.g., the lower layer is defined by the portion of the base within the edge region) and the folded layer(s) (e.g., 396a and 396b) of one of cuffs 340. Body 424 can be folded such that center region 428 has a width equal to separation distance 428 and each of edge regions 432a and 432b has a width equal to cuff width 360.

Additionally or alternatively, article 300b can comprise an underlayer 444 coupled to body 424. Underlayer 444 can comprise a nonwoven and can be configured to contain and prevent the SAP material each of cuffs 340 from escaping after the SAP material is wetted. Underlayer 444 may also maintain the structural integrity of tissue lamina(e) (e.g., 372a) after cuffs 340 absorb liquid. For example, underlayer 444 can be coupled to and span at least a portion of, optionally all of, a lower surface of base 440 that is defined by a portion of first body surface 436a. As shown, underlayer 444 spans across all of first body surface 436a such that the underlayer extends along the base and each of the folded layers. Underlayer 444 can facilitate securement of elastics strands 376 and 380. For example, underlayer 444 can extend from first body surface 436a and onto second body surface 436b, to which elastic strands 376 and 380 are coupled, such that the elastic strands are disposed between the second body surface and the underlayer. Underlayer 444 can be comparatively light to maintain cuff liftability; for example, the underlayer can comprise a nonwoven having a basis weight that is less than or equal to, or between any two of, 20 gsm, 18 gsm, 16 gsm, 14 gsm, 12 gsm, 10 gsm, 8 gsm, 6 gsm, or lighter (e.g., less than or equal to 10 gsm). Heavier basis weights can be used, e.g., to increase stiffness.

And, while article 300a has a topsheet that spans the lateral width of the absorbent core, topsheet 332 of article 300b can comprise first and second over-layers 448a and 448b, where each of the over-layers extends longitudinally along crotch portion 316 and is partially disposed on absorbent containment core 336 (e.g., along a respective longitudinal edge of the containment core) to define an uncovered portion 452 of the core therebetween. Over-layers 448a and 448b can comprise, for example, a liquid-permeable nonwoven, e.g., a nonwoven similar to that used for under layer 444.

Cuff assembly 420 can be attached to and disposed above absorbent core 336 such that base 440 at least partially covers uncovered portion 452. For example, center region 428 of body 424 can be attached to uncovered portion 452 via any suitable bond, such as with an adhesive 456 and/or with ultrasonic bonds. Adhesive 456 can be disposed as shown in FIG. 4A or can extend into each of edge regions 432a and 432b (e.g., to each of the outer longitudinal edges of cuffs 340). Containment core 336 can have substantially the same width as or can be narrower than cuff assembly 420; in other embodiments, however, the core can be wider than the cuff assembly. At least a portion of cuff assembly 420 (e.g., cuffs 340) can remain unattached to over-layers 448a and 448b, at least within free portion 388, such that the cuffs can lift away from chassis 304. In other embodiments, however, core 336 can be disposed above cuff assembly or “insert” 420, optionally such that the core is at least partially disposed beneath folded layers 396a and 396b of each of edge regions 432a and 432b and/or at least partially between the edge regions. In some of such embodiments, article 300b can include an acquisition distribution layer (e.g., a through-air bonded nonwoven) coupled to core 336. As shown, cuff assembly 420 is configured such that each of cuffs 340 is in a folded or “gasketing cuff” configuration; however, in other embodiments, each of the cuffs of the cuff assembly can be configured as a traditional “stand-up cuff” (e.g., an unfolded configuration).

Absorbent core 336 of article 300b can comprise one or more core laminates (e.g., 468a), each of which can be any of the laminates described above with respect to cuffs 340. The core laminate(s) can, but need not, be the same as the laminate from which cuffs 340 are constructed (if the cuffs are constructed from a laminate). As shown, for example, absorbent core 336 comprises a first laminate 468a that, similar to each of cuffs 340, has an absorbent lamina comprising SAP disposed between a first substrate lamina comprising tissue and a second substrate lamina comprising a nonwoven, where the second substrate lamina is disposed above both the absorbent lamina and the first substrate lamina (e.g., to facilitate acquisition of rapid insults of liquid). In other embodiments, however, absorbent core 336 can comprise any suitable number of core laminates layered on each other, such as, for example, greater than or equal to or between any two of 1, 2, 3, 4, 5, 6, or more core laminates (e.g., greater than or equal to 3 core laminates). Incorporating multiple core laminates can increase the amount of absorbent material (e.g., SAP) in absorbent core 336 and thereby increase the absorption capacity thereof.

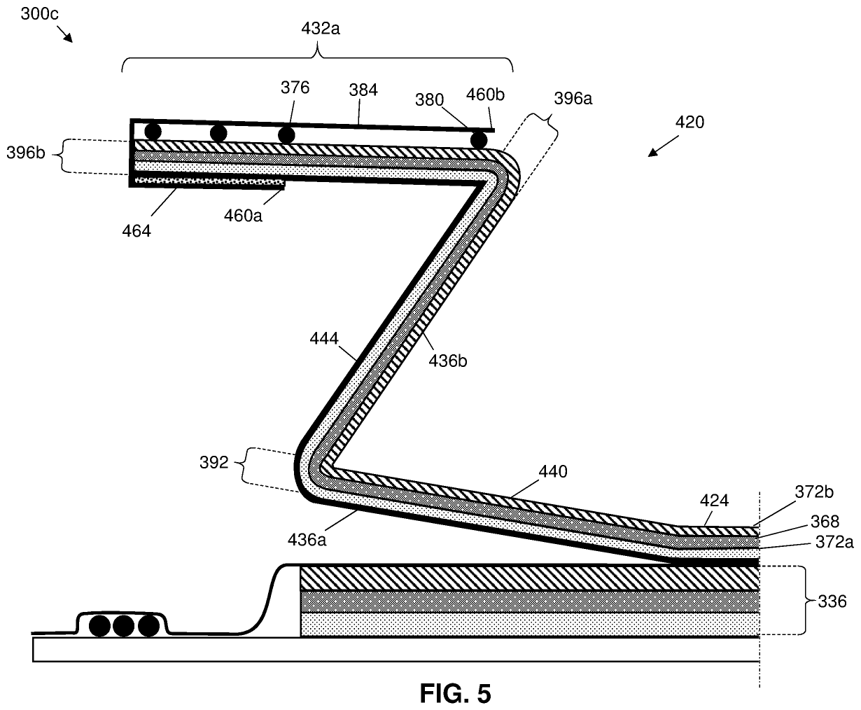

Referring to FIG. 5, shown is an absorbent article 300c that is substantially similar to article 300b, the primary exception being that underlayer 444 of article 300b does not extend onto second body surface 436b, and cover layer 384, rather than the underlayer, facilitates securement of elastic strands 376 and 380. For example, each cover layer 384 can have a first edge 460a coupled to first body surface 436a within a respective edge region (e.g., 432a or 432b) (e.g., to an uppermost one of the folded layer(s)) (e.g., via adhesive 464) and a second edge 460b coupled to second body surface 436b such that the cover layer extends from the first body surface onto the second body surface. Elastic strands 376 and 380 can thereby be disposed between second body surface 436b and cover layer 384. Cover layer 384 can be constructed from a nonwoven having a basis weight that is heavier than that of underlayer 444. Not to be bound by any particular theory, a comparatively lighter underlayer 444—which can span all of first body surface 436a—may facilitate lifting of cuffs 340 by reducing the weight thereof, and a comparatively heavier cover layer 384—which can be softer—may promote comfort when disposed on the skin-facing surface of each of the cuffs.

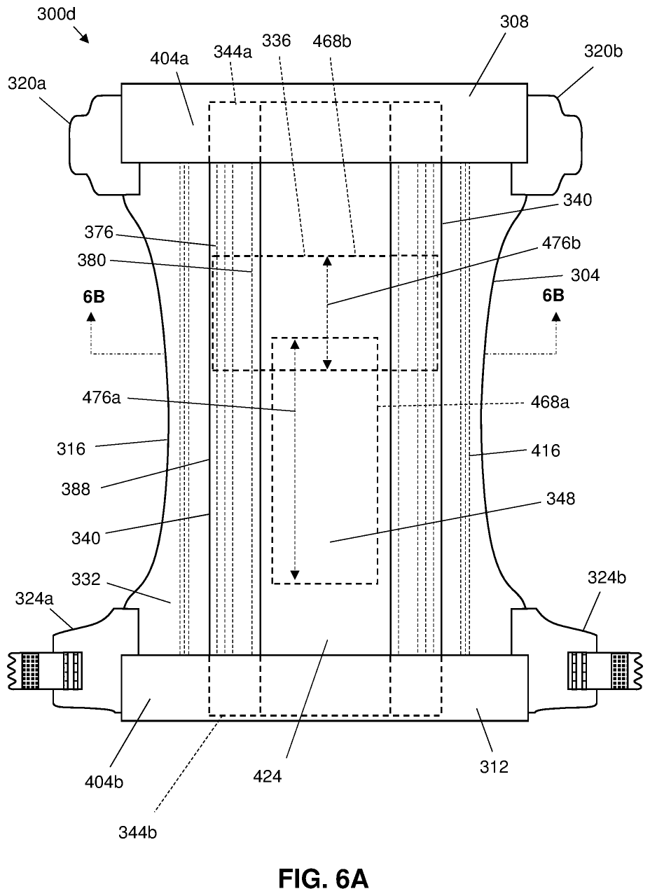

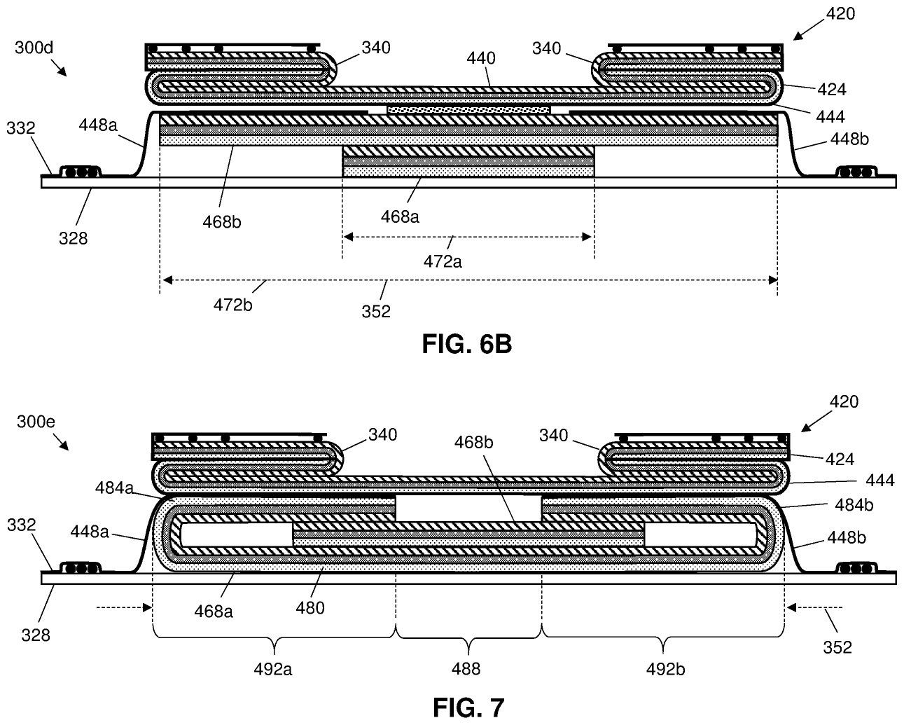

Referring to FIGS. 6A and 6B, shown is an absorbent article 300d that is substantially similar to article 300b, the primary exception being that absorbent core 336 of article 300d comprises two core laminates 468a and 468b. Core laminates 468a and 468b can be shaped and positioned relative to one another to further mitigate leakage from front portion 308 and/or rear portion 312. First and second core laminates 468a and 468b can have first and second lateral widths 472a and 472b, respectively, and first and second longitudinal lengths 476a and 476b, respectively. Second core laminate 468b can be wider than first core laminate 468a, e.g., second lateral width 472b can be at least, or between any two of, 10%, 20%, 30%, 40%, 50%, or more larger than first lateral width 472a. Additionally or alternatively, first core laminate 468a can be longer than second core laminate 468b, e.g., first longitudinal length 476a can be at least, or between any two of, 10%, 20%, 30%, 40%, 50%, or more larger than second longitudinal length 476b. To illustrate, first lateral width 472a can be less than or equal to, or between any two of, 120 mm, 100 mm, 80 mm, 60 mm, 40 mm, or less (e.g., less than or equal to 70 mm) and second lateral width 472b can be greater than or equal to, or between any two of, 60 mm, 80 mm, 100 mm, 120 mm, 140 mm, or more (e.g., greater than or equal to 70 mm). To further illustrate, first longitudinal length 476a can be greater than or equal to, or between any two of, 240 mm, 260 mm, 280 mm, 300 mm, 320 mm, 340 mm, or larger (e.g., greater than or equal to 280 mm) and second longitudinal length 476b can be less than or equal to, or between any two of, 240 mm, 220 mm, 200 mm, 180 mm, 160 mm, 140 mm, or less (e.g., less than or equal to 220 mm).

Second core laminate 468b can at least partially overlie or underlie first core laminate 468a and, optionally, can be positioned closer to front portion 308 than is the first core laminate (e.g., to mitigate leakage from the front portion). While article 300d has two core laminates 468a and 468b, in other embodiments the article can have more than two core laminates (e.g., two or more core laminates sized and positioned as described above with reference to first core laminate 468a and/or two or more core laminates sized and positioned as described above with reference to second core laminate 468b). And, as shown, cuffs 340 are disposed over at least a portion of second core laminate 468b but not first core laminate 468a; in other embodiments, however, the cuffs can be disposed over a portion of each of the first and second core laminates or can be positioned laterally outboard of both of the core laminates (e.g., depending on the core laminate widths and/or cuff separation). First and second core laminates 468a and 468b can optionally have different absorbent characteristics (e.g., different types or amounts of SAP). In other embodiments, any suitable number of core laminates can be used depending at least in part on the desired absorbent characteristics, such as, for example, greater than or equal to, or between any two of, 2, 3, 4, 5, 6, 7, or more core laminates.

Referring to FIG. 7, shown is an absorbent article 300e that is substantially similar to article 300b, the primary exception being that absorbent core 336 of article 300e comprises a folded first core laminate 468a. First core laminate 468a can be folded one or more times such that core 336 includes multiple laminate layers, including a lower layer 480 and one or more folded layers (e.g., 484a and 484b) disposed on the lower layer within each of first and second longitudinally-extending core edge regions 492a and 492b. Lower layer 480 can span lateral width 352 of core 336, and folded layers 484a and 484b can each span less than half of width 352 such that a gap is disposed between the folded layers. For example, each of core edge regions 492a and 492b can have a width that spans less than or equal to, or between any two of, 50%, 40%, 30%, 20%, or 10% (e.g., between 20% and 46%) of width 352. To illustrate, each of core edge regions 492a and 492b—and thus each of the folded layers—can have a width that is less than or equal to or between any two of, 70, 60, 50, 40, 30, 20, 10, or fewer millimeters (mm) (e.g., between 10 and 20 mm or between 30 and 65 mm). Each of core edge regions 492a and 492b can, but need not, be wider than each of cuffs 340.

Article 300e can also comprise a second core laminate 468b disposed on or under first core laminate 468a. Second core laminate 468b can have a lateral width and/or longitudinal length that is less than that of first core laminate 468a, and can be disposed on lower layer 480 such that at least a portion of the second core laminate is positioned laterally between folded layers 484a and 484b. Optionally, second core laminate 468b at least partially extends into each of core edge regions 492a and 492b such that, in each of the core edge regions, a portion of the second core laminate is disposed between lower layer 480 and the folded layer(s) defined therein. While, as shown, a single core laminate 468b is disposed on first core laminate 468a, in other embodiments two or more core laminates can be layered on the first core laminate.

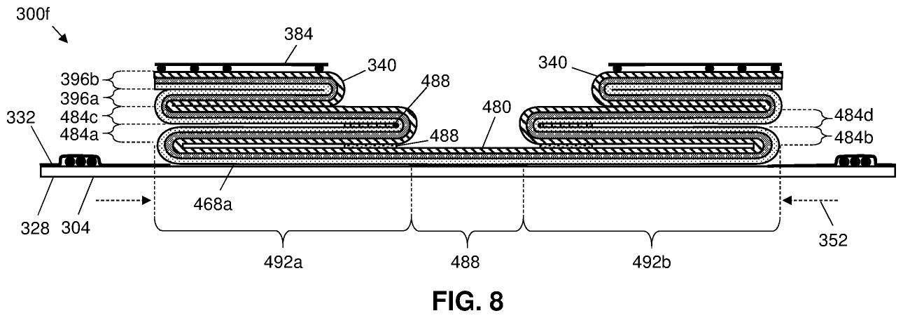

Referring to FIG. 8, shown is an absorbent article 300f that is substantially similar to article 300e, the primary exception being that in article 300f cuffs 340 are defined by and are integral with core laminate 468a instead of cuff assembly 420. An adhesive 488 can be disposed between one or more pairs of the layers (e.g., lower layer 480 and folded layers 484a-484d) of core laminate 468a to prevent the adhered layers from lifting away from chassis 304. One or more of the folded layers of core laminate 468a can define each of cuffs 340. For example, for each of cuffs 340, one or more of the folded layers of core laminate 468a can define folded layers 396a and 396b of the cuff, which can be disposed above the other folded layer(s) of the core laminate. The folded layer(s) of core laminate 468a that define each of cuffs 340 can be free of adhesive such that the cuffs can lift away from chassis 304. Topsheet 332 can optionally be configured such that the topsheet does not cover core laminate 468a and does not impede lifting of cuffs 340.

Absorbent articles 300a-300f, due at least in part to absorbent cuffs 340, can provide adequate leak prevention without the need for other acquisition and containment mechanisms, such as an acquisition distribution layer (ADL). Some of the present absorbent articles, however, may include such mechanisms to further improve leak prevention. For example, some of the present articles can, but need not, comprise an ADL (e.g., a through-air bonded nonwoven) to absorb rapid insults of liquid and reduce liquid accumulation. If used, an ADL can be disposed above cuff assembly 420 (optionally such that the ADL is at least partially underneath folded layers 396a and 396b of each of edge regions 432a and 432b) and/or above absorbent core 336.

The absorption before leakage (“ABL”) of some of the present absorbent articles (e.g., 300a-300f) (e.g., as measured according to the test method described in Example 2) can be greater than or equal to or between any two of 180 g, 190 g, 200 g, 210 g, 220 g, 230 g, 240 g, 250 g, 260 g, 270 g, 280 g or higher (e.g., between 200 and 280 g).

Some of the present methods of manufacturing an absorbent article (e.g., 300a-300f) comprise coupling first and second absorbent cuffs (e.g., 340) to a chassis (e.g., 304) having a crotch portion (e.g., 316) extending longitudinally between front and rear portions (e.g., 312). The chassis can have any of the components described above, such as an absorbent core (e.g., 336) disposed between a backsheet (e.g., 328) and a topsheet (e.g., 332). The coupling can be performed such that the first and second absorbent cuffs are sized and positioned on the chassis as described above with reference to any of articles 300a-300f.

Some methods comprise a step of forming a cuff assembly or insert (e.g., 420) that defines the cuffs. The cuff assembly or insert can be formed by folding an absorbent body (e.g., 424) that comprises an absorbent material, such as an absorbent laminate or a nonwoven containing superabsorbent material (e.g., any of the absorbent materials described above with reference to cuffs 340). The absorbent body can be folded one or more times such that the cuff assembly or insert defines a longitudinally-extending center region (e.g., 428) disposed between first and second longitudinally-extending edge regions (e.g., 432a and 432b), wherein the body defines the cuffs in the edge regions. For example, folding can be performed such that the cuff assembly or insert includes a base (e.g., 440) that spans a lateral width of the cuff assembly or insert and, within each of the edge regions: the body defines a lower layer (e.g., 392), which lower layer may be defined by the portion of the base within the edge region, and one or more folded layers (e.g., 396a and 396b) disposed above the lower layer. Each of the cuffs comprises the lower layer and the folded layer(s) defined in a respective one of the edge regions. Folding can be performed such that each of the cuffs comprises a Z-fold (e.g., as described with reference to article 300a), and such that the widths of the center region and edge regions—and thus the cuff width (e.g., 360) and cuff separation (e.g., 364)—are sized as described above with respect to articles 300a and 300b.

The body can comprise opposing first and second body surfaces (e.g., 436a and 436b). For example, when the body comprises a laminate that has an absorbent lamina (e.g., 368) comprising SAP disposed between a first substrate lamina (e.g., 372a) comprising tissue and a second substrate lamina (e.g., 372b) comprising a nonwoven, the tissue lamina can define the first body surface and the nonwoven lamina can define the second body surface. Some methods comprise crushing the SAP in the absorbent lamina, e.g., by applying pressure to the body with a roller. Before the folding, some methods comprise, for each of the cuffs, attaching one or more elastics (e.g., 376 and 380) to the second body surface (e.g., to the nonwoven lamina) such that the elastic(s) are disposed on an uppermost one of the folded layer(s) after the folding. Some methods also comprise, before the folding, coupling an underlayer (e.g., 444) to the first body surface, optionally such that the underlayer covers all of the first body surface. The underlayer, in some methods, can be larger than the first body surface such that at least a portion of the underlayer extends beyond each of the longitudinal edges (e.g., 400) of the body. Each of the portions of the underlayer that extend beyond the body can be folded over the longitudinal edge and secured to the second body surface such that the elastic(s) are disposed between the underlayer and the second body surface. The body can then be folded as described above to form the cuff assembly (e.g., of article 300b).

In some methods, the underlayer does not extend beyond the longitudinal edges of the body. Some of such methods comprise, for each of the longitudinal edges of the body, coupling a cover layer (e.g., 384) to the first body surface such that at least a portion of the cover layer extends beyond the longitudinal edge, folding the cover layer over the longitudinal edge, and securing the cover layer to the second body surface over the elastic(s) such that the elastic(s) are disposed between the cover layer and the second body surface. The body can then be folded as described above to form the cuff assembly (e.g., of article 300c).

In some methods, coupling the cuffs to the chassis comprises attaching the cuff assembly thereto (e.g., as described in reference to articles 300a-300e) after the cuff assembly is formed.

FIGS. 3A-3E, 4A-4B, 5, 6A-6B, 7, and 8 are exaggerated to better understand the overall structure of the present articles (e.g., 300a-300f), cuffs (e.g., 340), and absorbent cores (e.g., 336) and, as such, are for illustrative purpose only and are not necessarily to scale. For example, the figures illustrate the relative positions and relationships between elements of the present articles, including, for example, the position of laminae in a laminate (e.g., of cuff 340), the general folded structure of a cuff (e.g., 340), and the manner in which free portions of the cuff lift away from a chassis (e.g., 304), and should not be interpreted to limit the invention.

### EXAMPLES

The present invention will be described in greater detail by way of specific examples. The following examples are offered for illustrative purposes only and are not intended to limit the invention in any manner. Those of skill in the art will readily recognize a variety of non-critical parameters that can be changed or modified to yield essentially the same results.

### Example 1

An absorbency test was performed to compare the performance of one of the present absorbent cuff diapers with a conventional, Size 4 diaper that comprises hydrophobic cuffs. The absorbent cuff diaper was configured substantially the same as article 300b, except that the cuffs were positioned laterally outboard of the absorbent core, the core included two core laminates layered on one another, and there was no nonwoven underlayer coupled to the body of the cuff assembly. The absorbent core was 280 mm long and 60 mm wide and the cuffs were 380 mm long. The conventional diaper was obtained from Parent's Choice™.

For each of the diapers, the absorbency test was performed by placing the diaper on an inflatable baby mannequin and continuously introducing 350 ml of a 0.9% saline (sodium chloride) solution into the diaper via a tube inserted between the mannequin surface and the diaper. The conventional diaper leaked at the leg cuff before all of the solution was applied. The absorbent cuff diaper held all of the solution without leaks.

### Example 2

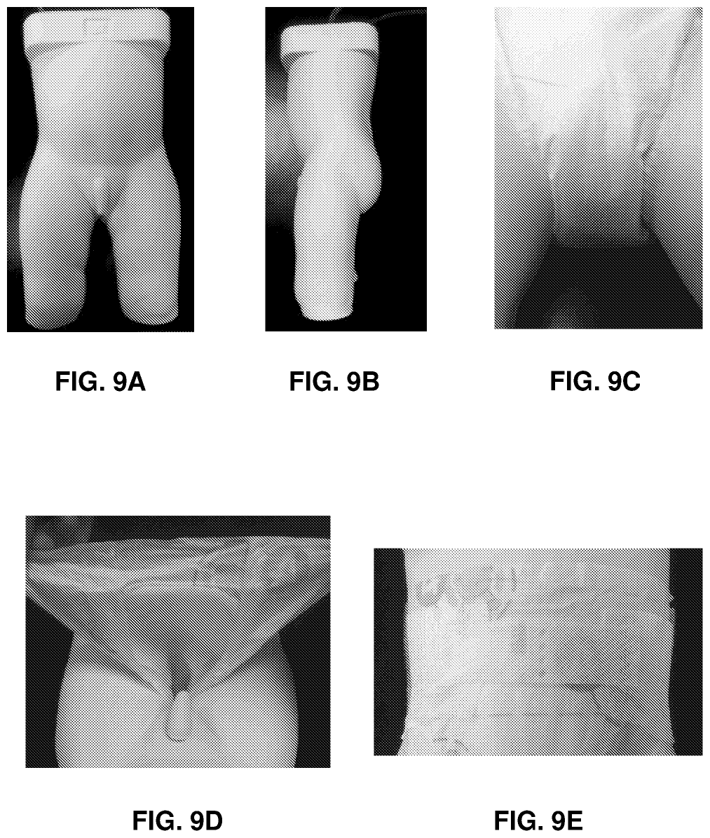

An absorbency-before-leakage test was performed to compare the performance of some of the present absorbent cuff diapers with that of a conventional diaper having a fluff/SAP absorbent core and hydrophobic cuffs (“Control”). During each test, the diaper was weighed to determine the initial dry weight thereof. The diaper was thereafter applied to a 360 mm-tall mannequin (FIGS. 9A and 9B) having a silicone rubber outer surface. The diaper was folded longitudinally to form a pouch between the legs of the mannequin (FIG. 9C) and was positioned such that the diaper's cuffs lifted to surround the male adapter on the mannequin (FIG. 9D). The leg elastics were adjusted such that they extended outwardly from the diaper (FIG. 9C). To secure the diaper to the mannequin, ears of the diaper were unfolded and fixed to a landing zone at the front portion thereof using tapes (FIG. 9E). A mattress was covered with a layer of tissue and the mannequin, with the diaper applied thereto, was placed onto the mattress with the mannequin's belly side facing downward. A pump was connected to the mannequin and set to introduce a 0.9% saline solution into the diaper via the male adapter at a rate of 420 ml per minute. During the first 10 minutes of the test, multiple doses of the saline solution were introduced into the diaper and the mannequin was flipped multiple times such that two doses were introduced while the mannequin's belly side faced downward and one dose was introduced while the mannequin's back side faced downward, as set forth below in TABLE 1. Each time the mannequin was flipped, the next dose was not introduced into the diaper until 1 minute after the flipping. If no leaks occurred during the initial 10-minute procedure, 25 ml doses of the saline solution were introduced every two minutes while the mannequin's belly side faced downward and until a leak occurred. Leakage was identified when the saline solution contacted the tissue and caused a dark spot to appear on the tissue.

TABLE 1
Initial 10-Minute Test Protocol for
the Absorbency-Before Leakage Test

| | Mannequin | | |
|---|---|---|---|
| Time (minutes) | Position | Dose Number | Volume (ml) |
| 0 | Belly | 1 | 75 |
| 4 | Back | — | — |
| 5 | Back | 2 | 25 |
| 9 | Belly | — | — |
| 10 | Belly | 3 | 75 |

When leakage occurred, the diaper was removed from the mannequin and weighed to determine the final wet weight thereof. The amount of liquid absorbed before leakage (“ABL”) was calculated by subtracting the initial dry weight of the diaper from the final wet weight. If multiple samples of the diaper were tested, the ABL was averaged.

The test was performed for the Control and five different absorbent cuff diapers (Versions A-E). Version A was substantially similar to absorbent article 300d, except that each of first and second absorbent laminates 468a and 468b were substantially the same size, a waistband was added at rear portion 312, and only rear end cap 404b included an elastic strip 408. Each of the core laminates had a width of 60 mm and a length of 280 mm and were constructed from NOVAZORB® 0417-TIP-SC50 75/75 HP500. The nonwoven substrate lamina comprised a 28 gsm 50% PET/50% viscose nonwoven. Version B was substantially similar to absorbent article 300d, except that a waistband was added at rear portion 312 and only rear end cap 404b included an elastic strip 408. In Version B, first core laminate 468a had a width of 60 mm and a length of 300 mm, second core laminate 468b had a width of 80 mm and a length of 200 mm, and cuff assembly 420 had a width of 80 mm and a length of 380 mm. Each of first and second core laminates 468a and 468b of Version B was constructed from NOVAZORB® 0417-TIP-SC50 II 75/75 T9900. Version C was substantially similar to Version B, except that each of first and second core laminates 468a and 468b used a different SAP material, e.g., the core laminates were constructed from NOVAZORB® 0417-TIP-SC50 III 75/75 HP 500. Version D was substantially similar to Version B, except that the absorbent laminate forming the cuff assembly was crushed using a hand roller before the diaper was constructed. Version E was substantially similar to Version D, except that the cuff assembly was not folded such that the cuffs of the cuff assembly were configured as a traditional “stand-up cuff.” Version F was substantially similar to Version B, except that it had no cuff assembly or cuffs. Version F instead had a laminate, which was the same laminate used for the cuff assembly of Version B, that covered the absorbent core, extended laterally to the edges of the impermeable film, and extended longitudinally such that it was 25 mm away from the edge of chassis 304 at front portion 308 and 25 mm away from the elastic band at rear portion 312. Three samples were tested for each of the Control and Versions B-E, and one sample was tested for each of Versions A and F.

The results of the absorbency-before-leakage test are set forth in TABLE 2. Each of the absorbent cuff designs had a higher average ABL than the standard hydrophobic cuff diaper. Of the designs tested, on average Versions F and C absorbed the most liquid before leaking.

TABLE 2
Absorbency-Before-Leakage Test Results

| | Sample | Initial Weight | Final Weight | ABL | Average |
|---|---|---|---|---|---|
| Product | Number | (g) | (g) | (g) | ABL (g) |
| Control | 1 | 31.29 | 202.68 | 171.39 | 146.13 |
| | 2 | 30.72 | 201.18 | 170.46 | |
| | 3 | 30.61 | 127.14 | 96.53 | |
| Version A | 1 | 33.97 | 279.46 | 245.49 | 245.49 |
| Version B | 1 | 31.15 | 204.14 | 172.99 | 199.46 |
| | 2 | 31.16 | 235.20 | 204.04 | |
| | 3 | 31.95 | 253.31 | 221.36 | |
| Version C | 1 | 31.83 | 310.94 | 279.11 | 257.91 |
| | 2 | 31.81 | 272.74 | 240.93 | |
| | 3 | 31.80 | 285.50 | 253.70 | |
| Version D | 1 | 31.94 | 229.71 | 197.77 | 224.60 |
| | 2 | 31.59 | 290.78 | 259.19 | |
| | 3 | 31.83 | 248.68 | 216.85 | |
| Version E | 1 | 29.84 | 198.23 | 168.39 | 210.21 |
| | 2 | 29.78 | 248.33 | 218.55 | |
| | 3 | 29.94 | 273.63 | 243.69 | |
| Version F | 1 | 28.09 | 298.05 | 269.96 | 269.96 |

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An absorbent article comprising:
a chassis having opposing front and rear portions and a crotch portion extending longitudinally between the front and rear portions;
an absorbent core extending longitudinally along the crotch portion; and
first and second absorbent cuffs, each:
separate from the other of the absorbent cuffs;
extending longitudinally along the crotch portion; and
positioned such that at least a portion of the absorbent core is positioned between the absorbent cuffs;
wherein each of the absorbent cuffs comprises:
a laminate having two or more substrate laminae and one or more absorbent laminae, a first one of the absorbent lamina(e) comprising superabsorbent polymer (SAP);
a liftable portion; and
one or more elastics configured to urge the liftable portion away from the chassis and toward a wearer when the absorbent article is worn.

2. An absorbent article comprising:
a chassis having opposing front and rear portions and a crotch portion extending longitudinally between the front and rear portions;
an absorbent core extending longitudinally along the crotch portion; and
a cuff assembly comprising an absorbent body that has opposing first and second body surfaces and defines first and second absorbent cuffs, each of the absorbent cuffs extending longitudinally along the crotch portion and positioned such that at least a portion of the absorbent core is positioned between the absorbent cuffs, wherein each of the absorbent cuffs comprises:
a liftable portion; and
one or more elastics configured to urge the liftable portion away from the chassis and toward a wearer when the absorbent article is worn.

3. The absorbent article of claim 2, wherein for each of the absorbent cuffs:
the absorbent cuff comprises a laminate having two or more substrate laminae and one or more absorbent laminae;
a first one of the absorbent lamina(e) comprises superabsorbent polymer (SAP) and is disposed between first and second ones of the substrate laminae; and
at least one of the first and second substrate laminae comprises a nonwoven.

4. The absorbent article of claim 3, wherein:
each of the absorbent cuffs is longitudinally folded such that the absorbent cuff includes a lower layer and one or more folded layers disposed above the lower layer
the absorbent body of the cuff assembly is longitudinally folded such that the cuff assembly:
includes a base that spans a lateral width of the cuff assembly and has a lower surface defined by a portion of the first body surface and an upper surface defined by a portion of the second body surface; and
defines a longitudinally-extending center region disposed between first and second longitudinally-extending edge regions, wherein within each of the edge regions, the body defines the lower layer and the folded layer(s) of one of the absorbent cuffs.

5. The absorbent article of claim 4, comprising the cuff assembly and a nonwoven underlayer coupled to the lower surface of the base.

6. The absorbent article of claim 5, wherein for each of the absorbent cuffs:
the elastic(s) are coupled to the second body surface; and
the underlayer extends along the first body surface and onto the second body surface such that the elastic(s) are disposed between the second body surface and the underlayer.

7. The absorbent article of claim 5, wherein for each of the absorbent cuffs:
the elastic(s) are coupled to the second body surface;
the absorbent article comprises a nonwoven cover layer having a first edge coupled to the first body surface in the edge region and a second edge coupled to the second body surface such that:
the cover layer extends along the first body surface and onto the second body surface; and
the elastic(s) are disposed between the second body surface and the cover layer; and
the underlayer has a first basis weight and the cover layer has a second basis weight heavier than the first basis weight.

8. The absorbent article of claim 2, comprising:
first and second nonwoven over-layers, each extending longitudinally along the crotch portion and partially disposed on the absorbent core such that an uncovered portion of the absorbent core is defined between the over-layers;
the center region of the cuff assembly is attached to the uncovered portion.

9. The absorbent article of claim 1, wherein for each of the absorbent cuffs:
the one or more elastics comprise a plurality of elastic strands extending longitudinally along the absorbent cuff, the elastic strands including two or more outer elastic strands and an inner elastic strand disposed closer to a longitudinal center axis of the absorbent article than are the outer elastic strands;
the outer elastic strands are laterally spaced apart from each other by a first separation distance; and
the inner elastic strand is laterally spaced apart from an innermost one of the outer elastic strands by a second separation distance that is at least 50% larger than the first separation distance, the innermost outer elastic strand disposed closer to the longitudinal center axis than are the other outer elastic strand(s).

10. The absorbent article of claim 1, wherein the absorbent core comprises one or more core laminates, each comprising:
two or more substrate laminae and one or more absorbent laminae;
wherein a first one of the absorbent lamina(e) comprises SAP and is disposed between first and second ones of the substrate lamina(e).

11. The absorbent article of claim 10, wherein:
the one or more core laminates comprise two or more core laminates;
a first one of the core laminates has a first length and a first width;
a second one of the core laminates has a second length shorter than the first length and a second width larger than the first width, wherein each of the lengths is measured longitudinally and each of the widths is measured laterally; and
the second core laminate at least partially overlies or underlies the first core laminate and is positioned closer to the front portion than is the first core laminate.

12. The absorbent article of claim 10, wherein:
the one or more core laminates comprise two or more core laminates;
a first one of the core laminates is longitudinally folded such that the absorbent core includes:
a lower layer of the first core laminate that spans a lateral width of the absorbent core; and
one or more folded layers of the first core laminate disposed above the lower layer within each of first and second longitudinally-extending core edge regions, each of the core edge regions spanning less than 50% of the width; and
a second one of the core laminates is disposed on the lower layer of the first core laminate.

13. The absorbent article of claim 1, wherein:
each of the absorbent cuffs extends longitudinally between opposing first and second ends; and
the absorbent article comprises first and second end caps, wherein:
the first end cap is attached to the front portion of the chassis and extends laterally across the first ends of the absorbent cuffs; and
the second end cap is attached to the rear portion of the chassis and extends laterally across the second ends of the absorbent cuffs.

14. The absorbent article of claim 13, wherein each of the first and second end caps comprises an absorbent material.

15. The absorbent article of claim 14, wherein for at least one of the first and second end caps, the end cap comprises:
a free portion that at least partially overlies and is unattached to the absorbent cuffs; and
one or more elastic strips, each configured to urge the free portion of the end cap away from the chassis and toward a wearer when the absorbent article is worn.

16. The absorbent article of claim 1, wherein for each of the absorbent cuffs:
the absorbent cuff comprises a laminate having two or more substrate laminae and one or more absorbent laminae;
a first one of the absorbent lamina(e) comprises superabsorbent polymer (SAP) and is disposed between first and second ones of the substrate laminae; and
at least one of the first and second substrate laminae comprises a nonwoven.

17. The absorbent article of claim 16, wherein:
each of the absorbent cuffs is longitudinally folded such that the absorbent cuff includes a lower layer and one or more folded layers disposed above the lower layer.

18. The absorbent article of claim 1, comprising:
first and second nonwoven over-layers, each extending longitudinally along the crotch portion and partially disposed on the absorbent core such that an uncovered portion of the absorbent core is defined between the over-layers.
